(19) United States
(12) Patent Application Publication
(10) Pub. No.: US 2011/0104268 A1
(43) Pub. Date: May 5, 2011

(54) GALENIC APPLICATIONS OF SELF-EMULSIFYING MIXTURES OF LIPIDIC EXCIPIENTS

(75) Inventors: Jean Pachot, La Varenne Saint Hilaire (FR); Serge Segot Chicq, L'Hay Les Roses (FR)

(73) Assignee: AVENTIS PHARMA S.A., Antony (FR)

(21) Appl. No.: 12/870,250

(22) Filed: Aug. 27, 2010

Related U.S. Application Data
(63) Continuation of application No. 11/572,402, filed on Dec. 19, 2007.

(30) Foreign Application Priority Data
Jul. 27, 2004 (FR) 0408269

(51) Int. Cl. A61K 9/48; A61K 9/14; A61K 31/505; A61P 35/00; C12N 5/09

(52) U.S. Cl. 424/452; 424/489; 514/256; 435/375

(57) ABSTRACT

A subject-matter of the invention is novel pharmaceutical formulations which make it possible to improve the intestinal absorption of orally administered active principles, their process of preparation and the application of lipid excipients in combination with one or more surfactants and one or more cosurfactants for inhibiting efflux pumps.

A subject-matter of the invention is novel pharmaceutical formulations which make it possible to improve the intestinal absorption of orally administered active principles, their process of preparation and the application of lipid excipients in combination with one or more surfactants and one or more cosurfactants for inhibiting efflux pumps.

Many active principles are weakly absorbed after oral administration.

A number of factors may be responsible for this poor absorption:

low solubility in the gastrointestinal medium in regions where the pH can vary between 5 and 8;

chemical or enzymatic decomposition of the active principle in the digestive tract;

efflux of the active principle at the intestinal epithelium via a pump, such as P-glycoprotein.

Numerous approaches for formulations have been provided in order to overcome the problems of absorption, these approaches being based either on modification of the physiology of the gastrointestinal route or on modification of the form of the medicament itself inside the digestive tract.

Generally, the increase in the absorption by a temporary modification of the characteristics of the gastrointestinal tract involves:

either the use of absorption promoters which act via a paracellular pathway by opening a tight junction (Liu, D. Z. et al., J. Pharm. Sci., 1999, 88 (11), 1161-1168; 1169-1174; Thanou, M. et al., J. Pharm. Sci., 2000, 90 (1), 38-46),

or additives which inhibit esterases in the gastrointestinal tract and thus enhance the stability of the prodrug (Van Gelder, J et al., Pharm. Res., 1999, 16 (7), 1035-1040; Van Gelder, et al., Drug Metab Dispo., 2000, 28 (12), 1394-1396),

or additives which modulate the transport of active compounds which is mediated by P-glycoprotein (Chang, T. et al., Clin Pharmacol Ther., 1996, 59 (3), 297-303; Zhang, Y. et al., Drug Metab Dispo., 1998, 26 (4), 360-366; Soldner, A. et al., Pharm. Res., 1999, 16 (4), 478-485).

An alternative strategy is to modify the disposition of the active principle inside the gastrointestinal tract. It is sufficient:

to increase the stability of compounds which are not very soluble in water by various methods which can be:
the use of solubilizing excipients (Saha, P. et al., Eur. J. Pharm. Biopharm., 2000, 50, 403-411),
the preparation of:
   solid dispersion formulations (Perng, C-Y et al., Int. J. Pharm., 1998, 176, 31-38; Chowdary, K. P. R. et al., Drug Dev. Ind. Pharm., 2000, 26 (11), 1207-1211),
   microemulsion formulations (Kommuru, T. R. et al., Int. J. Pharm., 2001, 212(2), 233-246; Pouton, C. V. et al., Eur. J. Pharm. Sci., 2000, 11, S93-S98; Gershanik, T. et al., Eur. J. Pharm. Biopharm., 2000, 50(1), 179-188),
   complexing formulations in cyclodextrin (Lin, H S et al., J. Clin. Pharm. Ther., 2000, 25(4), 265-269; Uekama, K. et al., J. Pharm. Sci., 1983, 72(11), 1338-1341),
to reduce the size of the particles (Farinha, A. et al., Drug Dev. Ind. Pharm., 2000, 26(5), 567-570),
to redirect the medicaments towards specific sites of the gastrointestinal tract in order to evade proteolysis of these medicaments by intestinal esterases (Bai, J P et al., Crit. Rev. Ther. Drug Carrier Syst., 1995, 12 (4), 339-371).

There still exists a need to find novel methods which make it possible to improve the intestinal absorption of medicaments which have received authorization for placing on the market or which are in the course of development. In particular, numerous medicaments exhibit a low oral bioavailability because they are substrates of pumps, such as P-glycoprotein. For this class of compounds, efflux by these pumps constitutes the limiting stage of the absorption process.

According to the present invention, the absorption of such active principles is significantly improved by the application of certain self-emulsifying mixtures of excipients which make it possible to inhibit efflux pumps. Novel pharmaceutical compositions comprising these mixtures have been employed according to the invention.

Self-emulsifying systems or SEEDS (Self Emulsifying Drug Delivery System) are solutions of oils and of surfactants which form oil-in-water emulsions or microemulsions when they are brought into the presence of an aqueous medium. When mixtures of lipid excipients and of surfactants and, if appropriate, of cosurfactants are incorporated in pharmaceutical compositions including active principles which are substrates of efflux pumps, emulsions or microemulsions are formed when these mixtures are in contact with an aqueous medium, such as the gastrointestinal fluid, and the efflux pumps are inhibited, which makes it possible to increase the intestinal absorption of the active principle. The invention thus applies very particularly to active principles known for being weakly absorbed after oral administration and for being substrates of efflux pumps.

This inhibition additionally results, if appropriate, in an increase in the solubility and/or the protection of the active principle against chemical decomposition in the digestive tract.

The result of the use according to the invention is a significant increase in intestinal absorption.

The type of formulation according to the invention also makes it possible to reduce the doses in comparison with a conventional formulation for the same therapeutic effectiveness, indeed even the same plasma exposure, which reduces the costs. The formulations according to the invention can also be applied to known and marketed active principles, thus making it possible to create novel pharmaceutical forms which exhibit an increased intestinal absorption or to extend a product conventionally administered parenterally (such as, for example, intravenously or subcutaneously) to application of the same active principle orally.

The mechanism for promoting intestinal passage is due to an interaction of the excipient according to the invention with the biological system rather than to an increase in the solubility. This is because, as is shown in the experimental part as described below, the absorption is less than 1% when the active principle is prepared in DMSO, for example, even though this is the solvent in which the solubility is the highest.

This mechanism has never been demonstrated with regard to the prior art. It makes it possible to envisage numerous possibilities for improving the oral bioavailability of active principles, the absorption of which is limited by the action of an efflux pump, such as P-glycoprotein.

The mechanism for promoting intestinal absorption of the systems according to the invention thus involves the inhibition of an efflux pump, such as P-glycoprotein. If appropriate, it also involves an increase in the solubility at the physiological pH values of the intestines and/or the protection against decomposition by digestive enzymes.

A subject-matter of the invention is thus the application of self-emulsifying mixtures of lipid excipients, of surfactants and, if appropriate, of cosurfactants, as defined below, in order to inhibit efflux pumps.

As is shown in the experimental tests described below, the lipid excipients, in combination with one or more surfactants and, if appropriate, one or more cosurfactants in a self-emulsifying mixture, act on one or more factors responsible for the poor absorption.

The pharmaceutical compositions according to the present invention thus make it possible to improve the intestinal absorption of active principles exhibiting one or more of the following parameters conflicting with optimum absorption:

low transepithelial passage in the direction of the absorption under the action of efflux pumps,

low solubility at the physiological pH values in the intestines,

chemical or enzymatic decomposition in the digestive tract.

A subject-matter of the invention is the application of self-emulsifying mixtures of lipid excipients, of surfactants and, if appropriate, of cosurfactants in the preparation of pharmaceutical compositions which can be administered orally including one or more active principles having the effect of enhancing the intestinal absorption of the said active principles by a mechanism involving inhibition of efflux pumps.

Another subject-matter of the invention is the application of self-emulsifying mixtures of lipid excipients, of surfactants and, if appropriate, of cosurfactants in the preparation of pharmaceutical compositions including one or more active principles having the effect of enhancing the intestinal absorption of the said active principles by a mechanism involving the inhibition of efflux pumps and an increase in the solubility of the active principle.

Another subject-matter of the invention is the application of self-emulsifying mixtures of lipid excipients, of surfactants and, if appropriate, of cosurfactants in the preparation of pharmaceutical compositions including one or more active principles having the effect of enhancing intestinal absorption of the said active principles by a mechanism involving inhibition of efflux pumps and an increase in the stability of the active principle in the gastrointestinal tract.

A further subject-matter of the invention is the application of self-emulsifying mixtures of lipid excipients, of surfactants and, if appropriate, of cosurfactants in the preparation of pharmaceutical compositions including one or more active principles having the effect of enhancing the intestinal absorption of the said active principles by a mechanism involving the inhibition of efflux pumps, an increase in the solubility of the active principle and an increase in the stability of the active principle in the gastrointestinal tract.

A more particular subject-matter of the invention is the use of self-emulsifying mixtures of lipid excipients, of surfactants and, if appropriate, of cosurfactants in order to inhibit the activity of P-glycoprotein.

According to the invention, the active principle is in particular picked up by P-glycoprotein and may be soluble or insoluble in the gastrointestinal tract or stable or unstable in the gastrointestinal tract.

The choice of the excipients and the choice of the ratios of these various excipients to one another is made in the following way: one of these excipients is an excipient of lipid nature and another excipient is a surfactant and/or another excipient is a cosurfactant, and these excipients are added in a ratio such that, for a given active principle, the mixture forms a self-emulsifying system.

The mixtures according to the invention can additionally comprise a solvent, such as glycofurol or DMSO.

The term “self-emulsifying system” is understood to mean a liquid or solid solution formed of a lipid excipient and optionally of a surfactant which can be lipophilic (that is to say, the hydrophilic/lipophilic balance [HLB] is greater than 10) or hydrophilic (HLB <10) and/or of a hydrophilic or lipophilic cosurfactant which forms oil-in-water emulsions, with particle sizes of between 0.1 and 10 μM, or oil-in-water microemulsions, with particle sizes of less than 100 nm, when it is added to an aqueous medium, directly or outside the physiological medium.

A subject-matter of the invention is preferably self-emulsifying mixtures of lipid excipients, of surfactants and, if appropriate, of cosurfactants which form oil-in-water microemulsions when they are added to an aqueous medium, directly or outside the physiological medium. According to the invention, the particles formed after interaction with an aqueous medium and in particular the duodenal fluid have a size of less than 100 nm.

The term “lipid excipient” is understood to mean in particular glycerides (mono-, di- and triglycerides), fatty acids and their derivatives, phospholipids, glycolipids and sterols.

According to the invention, the lipid excipients are chosen from glycerides, fatty acids and their derivatives, phospholipids, glycolipids and sterols.

The term “lipid excipient” is understood to mean, according to the invention, preferably:
glyceryl linoleate, such as Maisine 35-1® (Gattefossé),
glyceryl mono-oleate, such as Peceol® (Gattefossé),
glyceryl laurate, such as Gelucire 44/14® (macrogol-32) (Gattefossé),
glyceryl oleate/linoleate, such as Olicine®,
polyglyceryl-3 oleate, such as Plurol Oleique® (Gattefossé),
soybean oil,
capric/caprylic/lauric acid triglycerides, such as Captex 350® (Abitec Corporation), and
oleic acid.

The term “surfactant” is understood to mean an amphiphilic substance comprising two parts, one with a hydrophobic nature and the other with a hydrophilic nature, and which acts at a water/lipid or water/air interface by lowering the interfacial tension, even at low concentration. The surfactant is lipophilic if the HLB is greater than 10 and hydrophilic if it is less than 10.

According to the invention, the surfactant can in particular be hydrophilic.

According to the invention, the surfactant can be lipophilic, if appropriate.

According to the invention, the term “surfactant” is preferably understood to mean:
glyceryl caprylate/caprate, such as Labrasol® (macrogol-8) (Gattefossé),
polyoxyethylene-glycerol triricinoleate, such as Cremophor EL® (BASF), and
sorbitan polyoxyethylene oleate, such as Tween 80.

The term “cosurfactant” is understood to mean a substance which has the properties of a surfactant and which acts in the presence of a first surfactant by stabilizing the mixture formed by the surfactant and a lipid excipient.

The term “cosurfactant” is preferably understood to mean, according to the invention:
diethylene glycol monoethyl ether, such as Transcutol® (Gattefossé),
propylene glycol monocaprylate, such as Capryol 90® (Gattefossé),
absolute ethanol, and
macrogol 800 to 300.

According to the invention, the self-emulsifying mixtures of lipid excipients, of surfactants and, if appropriate, of cosurfactants are as follows:
System 1: Gelucire 44/14®/Plurol Oleique®/Transcutol®/DMSO, in proportions which can respectively vary between 50 and 60, 15 and 20, 15 and 20, and 5 and 15,
System 2: Gelucire 44/14®/Plurol Oleique®/Transcutol®/glycofurol, in proportions which can respectively vary between 50 and 65, 15 and 25, 15 and 25, and 5 and 15,
System 3: Gelucire 44/14®/Labrasol®/DMSO, in proportions which can respectively vary between 65 and 85, 15 and 25, and 5 and 15,
System 4: Gelucire 44/14®/Labrasol®/glycofurol, in proportions which can respectively vary between 65 and 85, 15 and 25, and 5 and 15,
System 5: Maisine 35-1®/Cremophor EL®/DMSO, in proportions which can respectively vary between 40 and 50, 40 and 50, and 5 and 15,
System 6: Maisine 35-1®/Cremophor EL®/glycofurol, in proportions which can respectively vary between 40 and 50, 40 and 50, and 5 and 15,
System 7: Soybean oil/Maisine 35-1®/Cremophor EL®/ethanol/DMSO, in proportions which can respectively vary between 25 and 35, 25 and 25, 25 and 35, 5 and 15, and 5 and 15,
System 8: Soybean oil/Maisine 35-1®/Cremophor EL®/ethanol/glycofurol, in proportions which can respectively vary between 25 and 35, 25 and 35, 25 and 35, 5 and 15, and 5 and 15,
System 9: Soybean oil/Maisine 35-1®/Cremophor EL®/Transcutol®/DMSO, in proportions which can respectively vary between 25 and 35, 25 and 35, 25 and 35, 5 and 15, and 5 and 15,
System 10: Soybean oil/Maisine 35-1®/Cremophor EL®/Transcutol®/glycofurol, in proportions which can respectively vary between 25 and 35, 25 and 35, 25 and 35, 5 and 15, and 5 and 15.

According to the invention, the self-emulsifying mixtures of lipid excipients, of surfactants and, if appropriate, of cosurfactants are in particular as follows:
System 1: Gelucire 44/14®/Labrasol®/DMSO in proportions 72/18/10;
System 2: Gelucire 44/14®/Labrasol®/glycofurol in proportions 72/18/10;
System 3: Gelucire 44/14®/Labrasol®/DMSO in proportions 80/20/10;
System 4: Gelucire 44/14®/Labrasol®/glycofurol in proportions 80/20/10;
System 5: Gelucire 44/14®/Plurol Oleique®/Transcutol®/DMSO in proportions 54/18/18/10;
System 6: Gelucire 44/14®/Plurol Oleique®/Transcutol®/glycofurol in proportions 54/18/18/10;
System 7: Maisine 35-1®/Cremophor EL®/DMSO in proportions 45/45/10;
System 8: Maisine 35-1®/Cremophor EL®/glycofurol in proportions 45/45/10;
System 9: Soybean oil/Maisine 35-1®/Cremophor EL®/ethanol/DMSO in proportions 27/27/28.8/7.2/10;
System 10: Soybean oil/Maisine 35-1®/Cremophor EL®/ethanol/glycofurol in proportions 27/27/28.8/7.2/10;
System 11: Soybean oil/Maisine 35-1®/Cremophor EL®/Transcutol®/DMSO in proportions 27/27/28.8/7.2/10;
System 12: Soybean oil/Maisine 35-1®/Cremophor EL®/Transcutol®/glycofurol: 27/27/28.8/7.2/10;
System 13: Soybean oil/Maisine 35-1®/Cremophor EL®/Transcutol®/DMSO in proportions 27.2/27.2/29.2/7.4/9;
System 14: Soybean oil/Maisine 35-1®/Cremophor EL®/Transcutol®/glycofurol in proportions 27.2/27.2/29.2/7.4/9;
System 15: Gelucire 44/14®/Plurol Oleique®/Transcutol®/glycofurol in proportions 55/18/18/9;
System 16: Gelucire 44/14®/Plurol Oleique®/Transcutol®/DMSO in proportions 55/18/18/9.

Another subject-matter of the invention is pharmaceutical compositions including an active principle and a self-emulsifying mixture of lipid excipients, of surfactants and, if appropriate, of cosurfactants as defined above.

As experimental examples, these systems were applied to active principles such as molecule A (ethyl ester of (2S)-2-(naphthyl-1-sulphonylamino)-3-(4-(2-(1,4,5,6-tetrahydropyrimidin-2-ylcarbamoyl)ethyl)-benzoylamino)propionic acid) or molecule B ((2S)-2-benzyloxycarbonylamino-3-(4-(3-(1,4,5,6-tetrahydropyrimidin-2-ylcarbamoyl)propyloxy)phenyl)-propionic acid), which are compounds of the family of the “Osteoclast Adhesion Receptor Antagonists” (O.A.R.A.) developed in the context of the prevention and treatment of osteoporosis, such as are defined in International Patent Applications WO 99/32457 and WO 99/37621.

The pharmaceutical compositions according to the invention are prepared in the following way:
1. Addition of the lipid excipient, of the surfactant and, if appropriate, of the cosurfactant. Semisolid excipients require preheating.
2. Mixing by stirring until a homogeneous solution is obtained.
3. Dissolution of the active principle in a solvent, such as DMSO, glycofurol or one of the excipients participating in the composition of the emulsions and of the microemulsions.
4. Addition of the dissolved active principle to the mixture of lipid excipient, surfactant and, if appropriate, cosurfactant.
5. If appropriate, heating or ultrasound treatment until a homogeneous solution is obtained.

The pharmaceutical compositions according to the invention can be provided in various forms, according to circumstances:
as a hard gelatin capsule filled with the semi-pasty, pasty or liquid mixture of excipients
as a soft capsule filled with the semi-pasty, pasty or liquid mixture of excipients
as a sealed vial filled with the liquid mixture of excipients
as a container of syrup bottle type filled with the liquid mixture of excipients.

In addition to their activity allowing the intestinal absorption to be enhanced, other advantages may be emphasized.

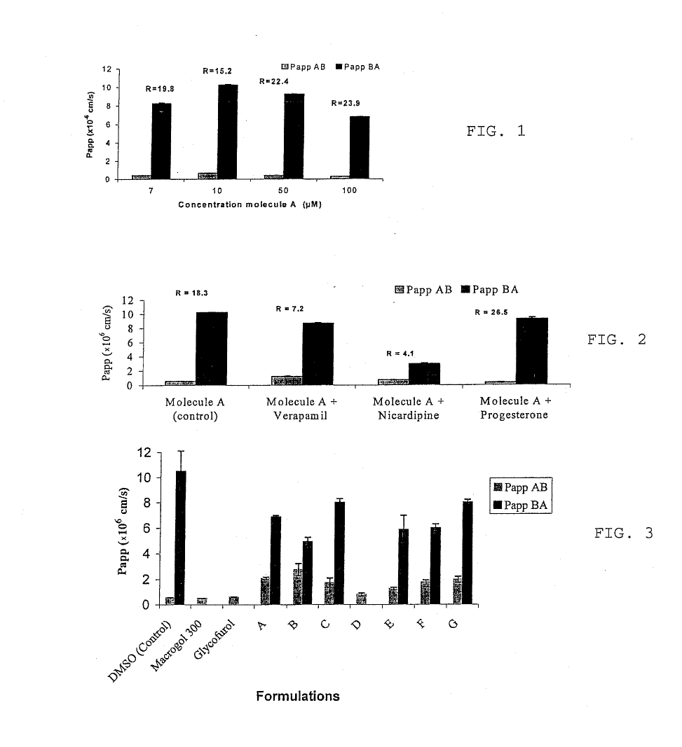

The formulations according to the invention make it possible to enhance the apparent permeability of an active principle in the AB direction (from the apical side towards the basolateral side) and to reduce that in the BA direction (from the basolateral side towards the apical side) in comparison with a control formulation (FIG. 1, Appendix 1).

The formulations according to the invention also make it possible to enhance the intracellular accumulation of an active principle in comparison with a control formulation (FIG. 4, Appendix 1).

Finally, they make it possible to stabilize an active principle in the intestinal fluid (for example duodenal fluid) by protecting the active principle from enzymatic hydrolysis (FIG. 5, Appendix 1).

Furthermore, some excipients according to the invention can be used by injection to inhibit the P-glycoprotein of cancer cells in order to enhance the cellular penetration of active principle into the tumour cells.

A subject-matter of the invention is thus the application of self-emulsifying mixtures of lipid excipients, of surfactants and, if appropriate, of cosurfactants in the preparation of an injectable solution which makes it possible to inhibit the P-glycoprotein of cancer cells and to enhance the cellular penetration of active principle into the tumour cells.

The following examples illustrate the invention without, however, limiting it.

### APPLICATIONAL EXAMPLES

### 1) Procedure

### 1.1) Molecules and Formulations Studied

### a) Molecule A: Formulations for In Vitro Study

The molecule A formulations for the in vitro study in the rat are shown in Table 1.

TABLE 1
Molecule A formulations used in the vitro study.

| Ingredients used at 1% in the | | |
|---|---|---|
| donor solutions | | |
| (HBSS/HEPES buffer) | Formulation | Composition |
| DMSO | DMSO | |
| Glycofurol | Glycofurol | |
| Macrogol 300 | Macrogol 300 | |
| Gelucire 44/14 ®/Labrasol ®/DMSO | A | 72/18/10 |
| Gelucire 44/14 ®/ | B | 54/18/18/10 |
| Plurol Oleique ®/Transcutol ®/ | | |
| DMSO | | |
| Gelucire 44/14 ®/Plurol Oleique ®/ | C | 54/18/18/10 |
| Transcutol ®/Glycofurol | | |
| Maisine 35-1 ®/ | D | 45/45/10 |
| Cremophor EL ®/DMSO | | |
| Soybean oil/Maisine 35-1 ®/ | E | 27/27/28.8/7.2/10 |
| Cremophor EL ®/Ethanol/DMSO | | |
| Soybean oil/Maisine 35-1 ®/ | F | 27/27/28.8/7.2/10 |
| Cremophor EL ®/Transcutol ®/ | | |
| DMSO | | |
| Soybean oil/Maisine 35-1 ®/ | G | 27/27/28.8/7.2/10 |
| Cremophor EL ®/Transcutol ®/ | | |
| Glycofurol | | |

### b) Molecule A: Formulation for the In Vivo Study

The molecule A formulations for the in vivo study in the rat are shown in Table 2.

TABLE 2
Molecule A formulations for the in vivo study in the rat.

| Formulation | Ingredients | Composition |
|---|---|---|
| Glyc/W | Glycofurol/Water | 50/50 |
| PEG | Macrogol 300/Water | 30/70 |
| Soy/Glyc | Soybean oil/Maisine 35-1 ®/ | 27.2/27.2/ |
| | Cremophor EL ®/Transcutol ®/ | 29.2/7.4/9 |
| | Glycofurol | |
| Gelu/Glyc | Gelucire 44/14 ®/ | 55/18/18/9 |
| | Plurol Oleique ®/Transcutol ®/ | |
| | Glycofurol | |
| Gelu/DMSO | Gelucire 44/14 ®/ | 55/18/18/9 |
| | Plurol Oleique ®/Transcutol ® / | |
| | DMSO | |

### c) Caco-2 Strains

The Caco-2 strains used in the tests are Caco-2/TC7 clone cells. This line is used to optimize the formulations and to investigate the mechanism or mechanisms of absorption in order to identify the parameter limiting the intestinal passage of active principles.

These cells were cultured and maintained according to methods known to a person skilled in the art.

### 1.2) Determination of the Solubility of Molecule a in Various Solvents

The solubility of molecule A is determined in purified water and in various buffers exhibiting pH values ranging between 1.2 and 8 (1.5, 2.5, 3.5, 4.5, 5.8, 6.8, 7.4 and 8.0).

10 mg of molecule A are added per 1 ml of aqueous solution.

The suspensions are stirred at 25° C. for 24 hours and are then centrifuged. The amount of molecule A in the supernatant is determined by HPLC and the pH of the supernatant is checked.

The apparent solubility of molecule A in the various oils, surfactants, cosurfactants, DMSO and glycofurol was also determined. Small amounts of molecule A are added to 1 g of each vehicle. Dissolution is carried out by ultrasound treatment at 25° C. Dissolution is confirmed visually and by optical microscopy. The solubility is estimated to within about 1 mg.

### 1.3) Preparation of the Formulations Tested in the Caco-2 Models and in the Rat:

a) Formulations Used to Study the Mechanism of Permeability of Molecule A with Regard to the Caco-2/TC7 Cell Models

In order to evaluate the scale of permeability of molecule A dissolved in DMSO as a function of the concentration, two solutions are prepared. The first, to which molecule A labelled with ¹⁴C will be added, is 4.3×10⁻²M; the second, to which ¹⁴C-mannitol will be added, is 5×10⁻²M.

These DMSO solutions are subsequently diluted in a 25 mM HBSS/HEPES buffer (pH 7.4) to which 0.4 μCi/ml of ¹⁴C-mannitol or 0.4 μCi/ml of molecule A labelled with ¹⁴C has been added (corresponding to 7 μM), so as to obtain final concentrations of molecule A of 7, 10, 50 or 100 μM.

The final concentration of DMSO in each donor solution is adjusted to 0.5%.

Donor solutions comprising 0.5% DMSO but comprising no compound are used as controls.

To analyse the role of P-glycoprotein in the mechanism of the transport of molecule A, donor solutions comprising 10 μM of molecule A and 100 μM of verapamil, nicardipine or progesterone are prepared and the permeability of molecule A is evaluated and compared with that obtained without the P-glycoprotein modulator.

### b) Effects of the Solvents Used on the Permeability in a Caco-2/TC7 Cell Model

To study the effects of glycofurol and of macrogol 300 on the permeability of molecule A:
Molecule A is dissolved in glycofurol in order to obtain 4.3×10⁻³M or 5×10⁻³M solutions. These are subsequently diluted in the HBSS/HEPES buffer to which 0.4 μCi/ml of ¹⁴C-mannitol or 0.4 μCi/ml of molecule A labelled with ¹⁴C has been added (see above), in order to obtain donor solutions for which the final concentration of molecule A is 50 μM and the final content of glycofurol is 1%.
Molecule A is dissolved in macrogol 300 in order to obtain 0.3×10⁻³M or 10⁻³M solutions. They are subsequently diluted in an HBSS/HEPES buffer to which 0.4 μCi/ml of ¹⁴C-mannitol or 0.4 μCi/ml of molecule A labelled with ¹⁴C have been added (see above), in order to obtain donor solutions for which the final concentration of molecule A is 50 μM and the final content of macrogol 300 in the donor solution is 5%.

The control donor solution comprising 0.5% DMSO and 5×10⁻⁵M of molecule A is prepared as indicated above.

### c) Effect of the Formulations on the Permeability of Molecule A in a Caco-2/TC7 Cell Model

The various formulations are prepared by mixing, under appropriate conditions, the lipid excipients, the surfactants and the cosurfactants, followed by vigorous stirring for 30 seconds (Table 1). When semisolid excipients are used, they are dissolved beforehand on a water bath at 50° C.

Before any formulation, molecule A is dissolved in DMSO or glycofurol in order to obtain, in each solvent, solutions with concentrations of 4.3×10⁻³M or 5×10⁻³M. 40 μCi/ml of molecule A labelled with ¹⁴C are added to the 4.3×10⁻³M solutions, so that the theoretical concentration of molecule A is 5×10⁻³M. 40 μCi/ml of ¹⁴C-mannitol are added to the 5×10⁻³M solutions.

Each of the solutions thus obtained is subsequently diluted in the mixtures under consideration of lipid excipients and of surfactants and, if appropriate, of cosurfactants, giving formulations comprising the solvent (DMSO or glycofurol) at 10% and molecule A at 5×10⁻⁴ M.

These formulations are diluted in 25 mM HBSS/HEPES buffer to give the donor solutions, the final concentration of molecule A of which is 5×10⁻⁵M, which comprise 0.4 μCi/ml of molecule A labelled with ¹⁴C or else 0.4 μCi/ml of ¹⁴C-mannitol, and the proportion of lipid excipient of which is less than 1%.

In order to evaluate the effects of the formulations, apical side, on the transport of mannitol and on the transport of molecule A in the BA direction, placebo solutions comprising the formulations but not molecule A were also prepared.

### d) Formulations Studied In Vivo in the Rat

1) Intravenous Administration

Molecule A labelled with ¹⁴C is injected in a 50/50 (v/v) glycofurol/water mixture at a concentration of 1.5 mg/ml (145.9 μCi/ml), which corresponds to the pharmacological dose. Glycofurol was chosen as the solvent which makes possible the administration of the desired amount of active principle, within the limits of the maximum volume which can be administered intravenously to the rat (1 ml/kg).

2) Oral Administration

The formulations are prepared as indicated in Table 2.

Molecule A labelled with ¹⁴C (220 μCi) is first dissolved in DMSO or glycofurol to produce solutions at a final concentration of 5 mg/ml (488.9 μCi/ml). These solutions are subsequently added to lipid mixtures in order to obtain the formulations described in Table 2, the final concentration of molecule A labelled with ¹⁴C being 0.45 mg/ml.

A control solution is prepared by dissolving molecule A labelled with ¹⁴C (220 μCi) in macrogol 300 at a final concentration of 0.5 mg/ml (44 μCi/ml).

Before any oral administration to the rat, the formulations are diluted in two volumes of water. The control solution of macrogol 300 is diluted in water, so as to obtain a final concentration of 0.15 mg/ml (13.2 μCi/ml). The formulations and the control thus prepared make it possible to administer, to the rat, 1.5 mg/kg in a volume of less than 10 ml/kg.

### 1.4) Study of the Transport

### a) The Cells and the Apparatus Used

In the transport studies, cells at passage 12 to 32 are deposited at a density of 5×10⁵ cells/filter on polycarbonate filters with a diameter of 12 mm in multiwell dishes (Transwell®, Costar). The cells are incubated at 37° C. for 21 to 28 days in complete medium supplemented with penicillin (100 IU/ml) and streptomycin (100 μg/ml) (Invitrogen).

A group of 6 wells is used to determine the permeability values of molecule A (in the AB or BA direction) for each solution given.

### b) The Formulations and the Solutions Used

When AB transport is studied, the basolateral medium is replaced with fresh HBSS/HEPES buffer (1.5 ml) and the apical medium (0.5 ml) with the donor solution.

When BA transport is studied, and with the exception of the lipid formulations described in Table 1, the apical medium is replaced with fresh HBSS/HEPES buffer and the basolateral medium with the donor solution. In order to study the effects of the lipid formulations on the permeability of molecule A in the BA direction, a control formulation of molecule A at 50 μM in an HBSS/HEPES buffer comprising 0.5% of DMSO is added on the basolateral side and a control solution is added on the apical side.

### c) Withdrawal and Treatment of the Samples

At T=0, 100 μl of the radioactive solution are withdrawn in order to quantify the initial radioactivity.

Every 30 min for 120 min, a 500 μl sample is withdrawn from the basolateral side and a 250 μl sample is withdrawn from the apical side for the study of the AB and BA transport respectively. The samples are immediately replaced with fresh HBSS/HEPES buffer or with the placebo formulation (in the case of experiments with lipid formulations in the BA direction).

The samples are measured by counting the β scintillation, after addition of a scintillation liquid, Aqueous Counting Scintillant (ACS, Amersham, Buckinghamshire, UK), with correction for quenching in simple labelling mode (LKB Wallac 1214, Broma, Sweden). For the studies of the transport in the AB direction with 7 μM and 100 μM of molecule A, the quantification is confirmed by LC/MS/MS.

### d) Confirmation of Membrane Integrity

Before each transport experiment, the confluence of the Caco-2 cells is confirmed by measuring the value of the transepithelial electrical resistance using an Endhom (WPI) equipped with planar electrodes. This value is of the order of 360 Ω·cm² for confluent monolayers of Caco-2 cells. Only confluent and differentiated Caco-2 cells are used for the transport experiments.

At the end of each transport study, the integrity of the monolayer is again confirmed by measuring the value of the transepithelial electrical resistance. The membrane integrity of the Caco-2 monolayer is regarded as being compromised when the value of the transepithelial electrical resistance decreases by more than 25% and when the apparent permeability to mannitol is greater than 10⁻⁶ cm/s.

### e) Calculation of the Flux

Under equilibrium conditions, the values of the unidirectional fluxes in the AB direction and the BA direction are calculated using the following equation:

J=dQ/dt×1/A

dQ representing the amount of active principle (counts/min) accumulated in the receiver compartment during the time interval dt and A being the exposed area of the monolayer (1.13 cm²).

### f) Calculation of the Apparent Permeability

The apparent permeability (Pₐₚₚ) of mannitol or of molecule A is obtained from the unidirectional flux by applying the following equation:

Pₐₚₚ=J/Cᵢ

Cᵢ is the initial number of counts/ml in the donor medium.

### g) Calculation of the Extrapolated Absorbed Fraction

The extrapolated absorbed fraction is calculated according to the equation (Pontier et al., J. Pharm. Sci., 2001, 90, 1608-1619):

$$Fa = \frac{0 - 100}{1 + {( \frac{log Papp}{- 5.595} )}^{- 24.064}} + 100$$

The extrapolated absorbed fraction is calculated for the studies of transport in the AB direction on the assumption that neither the solubility nor the degree of dissolution nor the efflux mechanism nor the stability in the gastrointestinal tract is a barrier for oral absorption.

### 1.5 Study of the Intracellular Concentration:

### a) Determination of the Flux and of the Intracellular Accumulation

The intracellular accumulation of molecule A is evaluated in parallel with studies of transport in the AB and BA directions using either a control donor formulation or a donor solution comprising formulation B, each of these formulations comprising molecule A labelled with ¹⁴C at 5×10⁻⁵M.

When formulation B is tested in the BA direction, the basolateral side comprises a control donor solution and the apical side is filled with the placebo of formulation B. As for the transport studies, the combined ingredients do not exceed 1% of the medium.

A total of 24 wells is used for each formulation, in each direction.

[1] Determination of the Fluxes

Samples of the medium are withdrawn at T=0, 30, 60, 120 and 180 minutes, either from the apical side or from the basolateral side, in order to determine the values of the fluxes (in DPM/cm²·h) in the AB and BA directions, as described above.

[2] Determination of the Intracellular Accumulation

In parallel, for each of these times, 6 wells are completely emptied of any medium and the corresponding filters are recovered and washed in PBS (Phosphate Buffered Saline) at 4° C.

These filters, which carry the Caco-2 cells, are introduced into a tube comprising 1 ml of a 50/50 (vol/vol) mixture of HBSS/HEPES buffer and of ethanol (95 vol %).

After resuspending the cells by ultrasound treatment for 1 min, the liquid is centrifuged at 1000 g for 5 min.

A 200 μl sample of supernatant is withdrawn and the radioactivity is counted with a scintillation counter.

The results are expressed in disintegrations per minute (DPM) accumulated in an apparent cell volume of 1 cm³. The following assumptions are made for the calculation of the volume of the monolayers: each cell forms a cylinder, the height of which is 17.9 μm and the diameter of which is 13.3 μm, and each monolayer comprises 1.1×10⁶ cells per cm², as has been reported (Pontier et al., J. Pharm. Sci., 2001 90, 1608-1619). The apparent volume of the monolayers growing on a 1.13 cm² polycarbonate filter is then 1.24×10⁻² cm³. At each time, the corresponding mean of the counts of the 6 wells (in DPM/cm³) is calculated.

### b) Evaluation of the Permeability Through the Apical and Basolateral Membranes

In order to calculate the values of apparent permeability from the intracellular compartment towards the basolateral side (CB) and from the intracellular compartment towards the apical side (CA), for the control formulation and for formulation B, it is assumed that the flux values obtained in the studies of transport in the AB and BA directions reflect a transfer of mass from the inside of the cells towards the outside, either from the basolateral side for the AB direction (J^{AB} being in DPM/cm²·h) or from the apical side for the BA direction (J^{BA} being in DPM/cm²·h).

It is also postulated that the J^{AB} and J^{BA} fluxes are both dependent on the intracellular concentrations C_{c}^{AB} and C_{c}^{BA} (expressed in DPM/cm³) calculated from the intracellular accumulation experiments carried out in parallel with the corresponding transport studies, in the AB and BA directions respectively. In this case, the fluxes measured in the AB direction (J^{AB}) and in the BA direction (J^{BA}) are equal to the fluxes from the inside towards the outside of the cell at the basolateral membrane (J^{CB}) and at the apical membrane (J^{CA}) respectively.

The membrane permeabilities are calculated according to the equations:

Pₐₚₚ^{CB}=J^{AB}/C_{c}^{AB}=J^{CB}/C_{c}^{AB}

and

Pₐₚₚ^{CA}=J^{BA}/C_{c}^{BA}=J^{CA}/C_{c}^{AB}

Pₐₚₚ^{CB} and Pₐₚₚ^{CA} are the mean membrane permeabilities in the CB and CA directions respectively.

The values of the mean membrane permeabilities are calculated using each of the 24 wells corresponding to the condition studied. The values of the mean fluxes and of the mean intracellular concentrations are also calculated using each of the 24 wells corresponding to the condition studied. The standard deviation of the population of the 24 wells is also calculated.

### 1.6) Stability of Molecule A in Human Duodenal Liquid

For each stability test, the necessary volume of a sample of human duodenal liquid frozen immediately after withdrawal is defrosted. Centrifuging at 1000 g for 15 min removes the substances resembling mucus. The pH of the supernatant is adjusted to 6.40 by addition of MES buffer (1250 mM in PBS-CMF), a value similar to the mean value of the pH of the fresh duodenal liquid.

Molecule A is dissolved in DMSO and either diluted directly in HBSS/HEPES buffer (control) or prepared in the formulations before dilution in HBSS/HEPES in order to obtain a microemulsion. The final concentration in both cases is 10⁻⁴M.

The formulations, preheated to 37° C., are added to the duodenal liquid, maintained at 37° C., in a 1/1 (v/v) ratio and are immediately mixed, in order for the final concentration of molecule A to be 5×10⁻⁵M.

At T=0 (immediately after mixing) and at T=5, 10, 15, 30, 60, 90 and 120 minutes, 100 μl samples of each preparation are withdrawn and mixed with the same volume of acetone at 4° C. to halt the enzymatic reaction. The samples were subsequently centrifuged (1000 g for 5 min) and the supernatant is tested by a validated LC/MS/MS method.

### 1.7) In Vivo Study

18 Sprague-Dawley rats (IFFA-Credo, St Germain sur l'Arbresle, France), each weighing 300-320 g and fed with a standard laboratory mixture (UAR 113, Villemoisson sur Orge, France), are used. After being deprived of food for 18 hours, they are divided into 6 equal groups, before receiving a dose of molecule A of 1.5 mg/kg, either orally (10 ml/kg) or intravenously (1 ml/kg).

The formulations used in the in vivo study are described in Table 2.

For oral administration, one volume of each of the three formulations tested is mixed with 2 volumes of water and vigorously stirred, in order to obtain a homogeneous emulsion comprising molecule A at a concentration of 0.15 mg/ml. The final concentration in each of these formulations is identical to that of the control formulation with PEG, that is to say 0.15 mg/ml (14.67 μCi/ml).

Each formulation is subsequently administered to four groups of rats by force feeding. The administration volume (10 ml/kg) is adjusted to the body weight in order to have a dose of 1.5 mg/kg. Two other groups of animals receive the control solution Glyc/w through the caudal vein at a dose of 1.5 mg/kg in a volume of 1 ml/kg.

For each of the groups which have received the formulation intravenously, the blood is collected by incision of the carotid artery at time 5 min (0.083 h). For all the other groups, the blood samples (0.2 ml) are collected at 0.25, 0.5, 1, 2 and 4 hours by retro-orbital withdrawal; at 6 hours, withdrawal is carried out by incision at the carotid artery.

The samples are collected over tubes treated with lithium heparinate and are stored at 4° C. The plasma is separated from the whole blood by centrifuging at 2000 g for 10 min at 4° C. The radioactivity present in the plasma fractions is measured with a scintillation counter. The concentration of molecule A labelled with ¹⁴C in the plasma is expressed in mg.eq/l.

The percentage of absorption (fₐ^{p.o}) of molecule A after oral administration is calculated as indicated below:

fₐ^{p.o.}=(AUC^{p.o}/AUC^{i.v}ₘₑₐₙ)×100

AUC^{p.o} is the area under the curve of concentration in the plasma from 0 to 6 hours after oral administration. AUC^{i.v}ₘₑₐₙ is the area under the curve of concentration in the plasma from 0 to 6 hours after intravenous administration.

For these calculations, it is assumed that the total clearance of the radioactivity is the same, whatever the route of administration of the product (orally or intravenously). The fraction absorbed is calculated for each animal and the mean and standard deviation are subsequently calculated for each group of animals receiving an oral formulation.

### 2) Results

### Example 1

### Molecule A

In a control formulation, molecule A is subjected to asymmetric transport with, depending on the concentration, Pₐₚₚ^{BA} from 15 to 24 times greater than Pₐₚₚ^{AB} (FIG. 1, Appendix 1). This effect is modulated by verapamil or nicardipine (FIG. 2, Appendix 1); it is thus due to the action of P-glycoprotein, which opposes the transepithelial passage in the direction of the absorption of molecule A.

Moreover, the solubility of molecule A is low (0.4 mg/ml) in an aqueous medium at a physiological pH of the intestines.

Finally, molecule A is unstable in human duodenal liquid (FIG. 5, Appendix 1).

The combination of these three factors—low permeation, low solubility, instability—results in a very low absorption of the active principle orally: less than 1% from a suspension.

### 1) Effect of the Formulations on the Action of P-Glycoprotein on the Transport of Molecule A

### a) Effect on the Transport of Molecule a Through the Caco-2 Monolayer

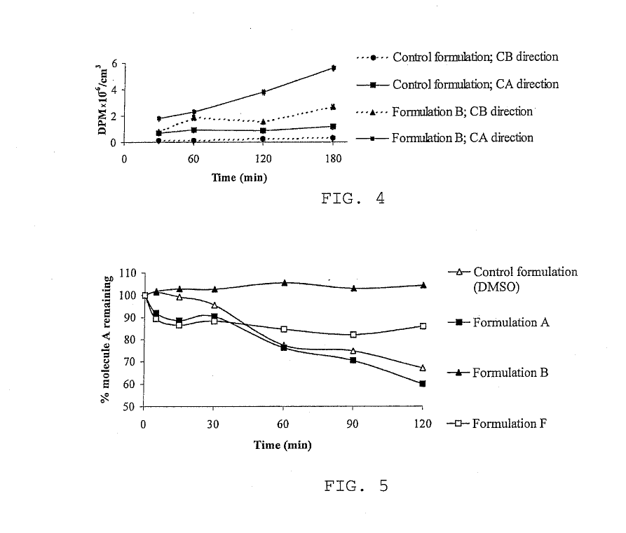

In experiments on transport through the monolayer where the formulations tested are in the donor solution, Pₐₚₚ^{BA} is reduced and Pₐₚₚ^{AB} is increased with respect to the control. The solvents used to prepare these formulations, glycofurol and macrogol 3000, have no effect on the Pₐₚₚ, values (FIG. 3, Appendix 1). The Pₐₚₚ^{BA}/Pₐₚₚ^{AB} ratio is only from 1.8 to 4.7, depending on the formulations used, whereas it is 18.3 for the control, indicating that the active efflux of molecule A is affected by the formulations.

b) Effect on the Intracellular Accumulation of Molecule A in the Caco-2 Monolayer

The intracellular accumulation of molecule A labelled with ¹⁴C and the flux through the cells were measured in parallel in experiments on transport through the Caco-2 monolayer.

When the apical compartment (that is to say, in contact with P-glycoprotein) comprises formulation B, the mean of the intracellular accumulation of molecule A labelled with ¹⁴C (C_{c}^{AB} and C_{c}^{BA}) increases with respect to the control, whatever the direction of the transport: it is greater by a factor of 8.5 in the AB direction and by a factor of 3.7 in the BA direction (Table 3).

When the apical compartment comprises a control solution (0.5% DMSO), Pₐₚₚ^{CA} is greater than Pₐₚₚ^{CB} by a factor of 4.2 because of the active transport of molecule A by P-glycoprotein. In contrast, in the presence of formulation B in the apical compartment, Pₐₚₚ^{CA} and Pₐₚₚ^{CB} are the same, indicating that active transport is inhibited.

TABLE 3
Intracellular accumulation and apparent permeability of molecule A.

| Formulation | Control | B |
|---|---|---|
| C_{c}^{AB} (DPM/cm³) | 2.0 × 10⁵ ± 2.6 × 10⁴ | 1.7 × 10⁶ ± 2.6 × 10⁵ |
| J^{CB} (DPM/cm² · h) | 1236 ± 98 | 8856 ± 1401 |
| Pₐₚₚ^{CB} (cm/s) | 1.7 × 10⁻⁶ ± 1.4 × 10⁻⁷ | 1.4 × 10⁻⁶ ± 2.3 × 10⁻⁷ |
| C_{c}^{BA} (DPM/cm³) | 9.2 × 10⁵ ± 5.3 × 10⁴ | 3.4 × 10⁶ ± 4.5 × 10⁵ |
| J^{CA} (DPM/cm² · h) | 23 625 ± 2630 | 16 000 ± 783 |
| Pₐₚₚ^{CA} (cm/s) | 7.1 × 10⁻⁶ ± 7.9 × 10⁻⁷ | 1.3 × 10⁻⁶ ± 8.2 × 10⁻⁸ |

### 2) Effect of the Formulations on the Solubility of Molecule A

The solubility of molecule A in aqueous solutions is very low at physiological pH (0.4 mg/ml). In glycofurol and macrogol 300, used in the formulations tested, the solubility of molecule A is 6 mg/ml and 2 mg/ml respectively.

### 3) Effect of the Formulations on the Stability of Molecule A

The stability of molecule A in human duodenal liquid was measured. In a control formulation, 30% of the active principle is hydrolysed after 120 minutes. In contrast, at the same time, 100% and 85% of molecule A are still present with formulations B and F respectively.

### 4) Effect of the Formulations on the Absorption of Molecule A

Molecule A was given orally to rats in various formulations (Table 2). In a solvent system such as PEG, the absorption is only 25%. The absorption is 100% for each of the three formulations used (Table 4).

TABLE 4
Percentage of absorption (fₐ^{p.o}) after oral administration
of the formulations to the rat.

| | AUC (0-6 h) | |
|---|---|---|
| | (mq · eq/ml) ± sd | |
| Formulation | (CV %) | fₐ^{p.o.} |
| Water/glycofurol: | 0.68 ± 0.13 (32%) | — |
| 50/50 (i.v.) | | |
| PEG | 0.173 ± 0.046 (46%) | 25.4% ± 6.8 |
| Soy/Glyc | 0.76 ± 0.16 (37%) | 111% ± 24 |
| Gelu/Glyc | 1.00 ± 0.11 (19%) | 147 ± 16 |
| Gelu/DMSO | 0.94 ± 0.11 (21%) | 138% ± 17 |

### 5) Molecule A: Summary of the Results

The result show that the formulations according to the invention make it possible:
to increase the apparent permeability in the AB direction and to reduce that in the BA direction with respect to a control formulation (FIG. 3, Appendix 1)
to increase the intracellular accumulation of molecule A with respect to a control formulation (FIG. 4, Appendix 1)
to stabilize molecule A in the duodenal fluid by protecting the active principle from enzymatic hydrolysis (FIG. 5, Appendix 1)
to obtain complete absorption in the animal, whereas it is only 25% in a solvent system such as PEG 300 and whereas the absolute bioavailability is less than 1% from a suspension (Table 4).

### Example 2

### Molecule B

Results for transport through the Caco-2 monolayers show that molecule B undergoes efflux by P-glycoprotein. This is because, in a formulation comprising 0.5% of DMSO, the following results are obtained:

Pₐₚₚ in the AB direction: 4.4×10⁻⁷ cm/s

Pₐₚₚ in the BA direction: 2.1×10⁻⁶ cm/s

A formulation including 1.7% of Gelucire 44/14®/Labrasol® in the proportions 80/20 in the transport medium makes it possible to modulate the passage of molecule B through the Caco-2 monolayers in the following way:

Increase in the Transport in the Ab Direction (direction of the absorption) by a factor of 5.9 with respect to a medium including 0.5% of DMSO, the apparent permeability (Pₐₚₚ) changing from 4.4×10⁻⁷ cm/s to 2.6×10⁻⁶ cm/s.

FIG. 1

Permeability of molecule A in the two directions (AB and BA) in monolayers of Caco-2 cells.

FIG. 2

Modulation of the permeability of molecule A through the Caco-2 cell monolayers in the two directions AB and BA by verapamil, nicardipine and progesterone at 100 μM in the donor solution.

FIG. 3

Effect of various formulations on the permeability of molecule A through the monolayers of Caco-2 cells in the two directions AB and BA.

FIG. 4

Intracellular accumulation of ¹⁴C-molecule A (50 μM) in the two directions AB and BA with a control formulation (0.5% DMSO) and formulation B.

FIG. 5

Stability in human duodenal liquid of molecule A formulated in DMSO or in formulations A, B or F.

1. A method of enhancing the absorption of an active principle by a mechanism involving inhibition of efflux pumps, comprising orally administering a pharmaceutical composition, wherein the pharmaceutical composition comprises:
a self-emulsifying mixture, said self-emulsifying mixture comprising
one or more lipid excipients in an amount of 40 to 85%, the one or more lipid excipients being selected from the group consisting of glyceryl linoleate, glyceryl mono-oleate, glyceryl oleate/linoleate, glyceryl laurate, polyglyceryl-3 oleate, soybean oil, capric/caprylic/lauric acid triglycerides, and oleic acid,
one or more surfactants in an amount of 15 to 50%, and
optionally one or more cosurfactants; and
one or more active principles.

2. A method according to claim 1, characterized in that the self-emulsifying mixture additionally comprises a solvent.

3. A method according to claim 1, characterized in that the composition enhances the absorption of the active principle by a mechanism involving:
inhibition of efflux pumps and
increase in the solubility of the one or more active principles.

4. A method according to claim 1, characterized in that the composition enhances the absorption of the one or more active principles by a mechanism involving:
inhibition of efflux pumps and
increase in the stability of the one or more active principles in the gastrointestinal tract.

5. A method according to claim 1, characterized in that the composition enhances the absorption of the active principle by a mechanism involving:
inhibition of efflux pumps,
increase in the solubility of the one or more active principles and
increase in the stability of the one or more active principle principles in the gastrointestinal tract.

6. A method according to claim 1, wherein the efflux pump is P-glycoprotein.

7. A method according to claim 1, wherein said self-emulsifying mixtures form oil-in-water micro emulsions, with particle sizes of less than 100 nm after interaction with an aqueous medium, and in particular the duodenal fluid.

8. (canceled)

9. (canceled)

10. A method according to claim 1, wherein the one or more surfactants are hydrophilic.

11. A method according to claim 1, wherein the one or more surfactants are lipophilic.

12. A method according to claim 1, wherein the one or more surfactants are selected from the group consisting of:
glyceryl caprylate/caprate,
polyoxyethylene-glycerol triricinoleate, and
sorbitan polyoxyethylene oleate.

13. A method according to claim 1, wherein the self-emulsifying mixture comprises one or more cosurfactants, and the one or more cosurfactants are selected from the group consisting of:
diethylene glycol monoethyl ether,
propylene glycol monocaprylate,
absolute ethanol, and
macrogol 800 to 300.

14. A method according to claim 1, wherein the self-emulsifying mixture is composed of glyceryl laurate/polyglyceryl-3 oleate/diethylene glycol monoethyl ether/DMSO, in proportions which can respectively vary between 50 and 60, 15 and 20, 15 and 20, and 5 and 15.

15. A method according to claim 1, wherein the self-emulsifying mixture is composed of glyceryl laurate/polyglyceryl-3 oleate/diethylene glycol monoethyl ether/glycofurol, in proportions which can respectively vary between 50 and 65, 15 and 25, 15 and 25, and 5 and 15.

16. A method according to claim 1, wherein the self-emulsifying mixture is composed of glyceryl laurate/macrogol-8/DMSO, in proportions which can respectively vary between 65 and 85, 15 and 25, and 5 and 15.

17. A method according to claim 1, wherein the self-emulsifying mixture is composed of glyceryl linoleate/polyoxyethylene-glycerol triricinoleate/DMSO, in proportions which can respectively vary between 40 and 50, 40 and 50, and 5 and 15.

18. A method according to claim 1, wherein the self-emulsifying mixture is composed of glyceryl laurate/macrogol-8/glycofurol, in proportions which can respectively vary between 65 and 85, 15 and 25, and 5 and 15.

19. A method according to claim 1, wherein the self-emulsifying mixture is composed of glyceryl linoleate/polyoxyethylene-glycerol triricinoleate/glycofurol, in proportions which can respectively vary between 40 and 50, 40 and 50, and 5 and 15.

20. A method according to claim 1, wherein the self-emulsifying mixture is composed of soybean oil/glyceryl linoleate/polyoxyethylene-glycerol triricinoleate/ethanol/DMSO, in proportions which can respectively vary between 25 and 35, 25 and 35, 25 and 35, 5 and 15, and 5 and 15.

21. A method according to claim 1, wherein the self-emulsifying mixture is composed of soybean oil/glyceryl linoleate/polyoxyethylene-glycerol triricinoleate/ethanol/glycofurol, in proportions which can respectively vary between 25 and 35, 25 and 35, 25 and 35, 5 and 15, and 5 and 15.

22. A method according to claim 1, wherein the self-emulsifying mixture is composed of soybean oil/glyceryl linoleate/polyoxyethylene-glycerol triricinoleate/diethylene glycol monoethyl ether/DMSO, in proportions which can respectively vary between 25 and 35, 25 and 35, 25 and 35, 5 and 15, and 5 and 15.

23. A method according to claim 1, wherein the self-emulsifying mixture is composed of soybean oil/glyceryl linoleate/polyoxyethylene-glycerol triricinoleate/diethylene glycol mono ethyl ether/glycofurol, in proportions which can respectively vary between 25 and 35, 25 and 35, 25 and 35, 5 and 15, and 5 and 15.

24. Pharmaceutical composition comprising:
an active principle;
a self-emulsifying mixture (SEEDS) of one or more lipid excipients in an amount of 40 to 85%, the one or more lipid excipients being selected from the group consisting of glyceryl linoleate, glyceryl mono-oleate, glyceryl oleate/linoleate, glyceryl laurate, polyglyceryl-3 oleate, soybean oil, capric/caprylic/lauric acid triglycerides, and oleic acid; and
one or more surfactants in an amount of 15 to 50%; and
optionally comprising one or more co-surfactants.

25. Pharmaceutical composition according to claim 24 wherein the surfactants are selected from the group consisting of:
glyceryl caprylate/caprate
polyoxyethylene-glycerol triricinoleate, and
sorbitan polyoxyethylene oleate.

26. Pharmaceutical composition according to claim 24, characterized in that it exists as a hard gelatin capsule filled with the semi-pasty, pasty or liquid mixture of excipients.

27. Pharmaceutical composition according to claim 24, characterized in that it exists as a soft capsule filled with the semi-pasty, pasty or liquid mixture of excipients.

28. Pharmaceutical composition according to claim 24, characterized in that it exists as a sealed vial filled with the liquid mixture of excipients.

29. Pharmaceutical composition according to claim 24, characterized in that it exists as a container of syrup bottle type filled with the liquid mixture of excipients.

30. Pharmaceutical composition according to claim 24, characterized in that the active principle is the ethyl ester of (2S)-2-(naphthyl-1-sulphonylamino)-3-(4-(2-(1,4,5,6-tetrahydropyrimidin-2-ylcarbamoyl)ethyl)benzoylamino)-propionic acid.

31. (canceled)

32. Pharmaceutical composition according to claim 24, characterized in that the active principle is (2S)-2-benzyloxycarbonyl amino-3-(4-(3-(1,4,5,6-tetrahydropyrimidin-2-ylcarbamoyl)propyloxy)phenyl)-propionic acid.

33. Pharmaceutical composition according to claim 25, characterized in that the mixture comprises glyceryl laurate/macrogol-8 in proportions 80/20.

34. A method for inhibiting to P-glycoprotein of cancer cells and to enhance the cellular penetration of the active principle into the tumour cells comprising administering by injection an injectable solution that inhibits P-glycoprotein of cancer cells and enhances the cellular penetration of the active principle into the tumour cells;
wherein the injection solution comprises a self-emulsifying mixture comprising one or more lipid excipients, one or more surfactants, and, optionally, one or more cosurfactants.

35. Process for the preparation of self-emulsifying mixtures (SEEDS), the self emulsifying mixtures comprising:
one or more lipid excipients in an amount of 40 to 85%, the one or more lipid excipients being selected from the group consisting of glyceryl linoleate, glyceryl mono-oleate, glyceryl oleate/linoleate, glyceryl laurate, polyglyceryl-3 oleate, soybean oil, capric/caprylic/lauric acid triglycerides, and oleic acid,
one or more surfactants in an amount of 15 to 50%, and
optionally one or more cosurfactants,
the process comprising:
adding of the lipid excipient, of the surfactant and, if appropriate, of the cosurfactant, semisolid excipients requiring preheating;
mixing by stirring until a homogeneous solution is obtained;
dissolving the active principle in a solvent, such as DMSO, glycofurol or one of the excipients participating in the composition of the emulsions and of the microemulsions;
adding the dissolved active principle to the mixture of lipid excipient, surfactant and, if appropriate, cosurfactant;
if appropriate, performing heating or ultrasound treatment until a homogeneous solution is obtained.

36. A method according to claim 1, characterized in that the self-emulsifying mixture additionally comprises a solvent which is DMSO or glycofurol.

37. Pharmaceutical composition according to claim 24, wherein the surfactants are selected from the group consisting of:
glyceryl caprylate/caprate,
polyoxyethylene-glycerol triricinoleate, and
sorbitan polyoxyethylene oleate.
and the composition comprises one or more cosurfactants, and the one or more cosurfactants are selected from the group consisting of:
diethylene glycol monoethyl ether,
propylene glycol monocaprylate,
absolute ethanol, and
macrogol 800 to 300.

38. Pharmaceutical composition according to claim 24, characterized in that the mixture is composed of glyceryl laurate/polyglyceryl-3-oleate/Diethylene glycol mono ethyl ether/DMSO in proportions 54/18/18/10.
